(19) United States
(12) Patent Application Publication
(10) Pub. No.: US 2025/0270334 A1
(43) Pub. Date: Aug. 28, 2025

(54) ANTl-CMPL DIVALENT SCFV AND USES THEREOF

(71) Applicant: THE UNITED STATES OF AMERICA, as represented by the Secretary, Department of Health and Human, Bethesda, MD (US)

(72) Inventors: Andre Larochelle, Bethesda, MD (US); Daisuke Araki, Bethesda, MD (US); Diogo Magnani, Boston, MA (US); Zhirui Wang, Boston, MA (US)

(21) Appl. No.: 18/688,899

(22) PCT Filed: Sep. 30, 2022
(86) PCT No.: PCT/US2022/077326

§ 371 Date: Aug. 22, 2024

Related U.S. Application Data
(60) Provisional application No. 63/251,883, filed on Oct. 4, 2021.

(51) Int. Cl. C07K 16/28; A61P 35/02; C12N 9/10

(52) U.S. Cl. CPC C07K 16/2866; A61P 35/02; C12N 9/1077; C12Y 204/02036; C07K 2317/35; C07K 2317/622

(57) ABSTRACT

This disclosure relates to immunotoxin fusion proteins that comprise a bivalent single-chain fragment variable that specifically binds to the thrombopoietin receptor (cMPL); and a diphtheria toxin. The disclosure also provides compositions comprising such proteins and nucleic acid molecules encoding such proteins and uses thereof.

### CROSS REFERENCE TO RELATED APPLICATIONS

This application claims the benefit of U.S. Provisional Application No. 63/251,883, filed Oct. 4, 2021, which is incorporated by reference herein in its entirety.

### STATEMENT REGARDING FEDERALLY SPONSORED RESEARCH

This project was made with government support by the National Institutes of Health Intramural Research Program grants Z99 HL999999 and ZIA HL006172, and Extramural Research Program grant P40OD028116. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Sep. 27, 2022, having the file name “21-1209-WO_Sequence-Listing.xml” and is 25 kilobytes in size.

### FIELD OF THE DISCLOSURE

This disclosure generally relates to immunotoxin fusion proteins that comprise a bivalent single-chain fragment variable that specifically binds to the thrombopoietin receptor (cMPL) and a diphtheria toxin. The disclosure also provides compositions comprising such proteins and nucleic acid molecules encoding such proteins and uses thereof.

### BACKGROUND

Patient conditioning is a critical initial step in hematopoietic stem and progenitor cell (HSPC) transplantation procedures to enable marrow engraftment of infused cells. Preparative regimens have traditionally been achieved by delivering cytotoxic doses of chemotherapeutic agents, with or without radiation. However, these regimens impair host immune function and are associated with significant morbidity. The use of monoclonal antibodies, either alone or conjugated to an internalizing toxin, to target specific antigens on hematopoietic cells has been proposed as a tractable alternative, especially in contexts, such as ex vivo autologous gene therapy, where preservation of immunity is desired. Efficient clearance of marrow has been demonstrated in preclinical models using CD45- or CD117-targeting antibodies conjugated to the plant toxin Saporin. However, this approach still awaits demonstration of long-term safety and efficacy in humans. Thus, there remains a need in the art to establish a nontoxic preparative approach to improve HSPC engraftment in transplantation for genetic and other nonmalignant disorders. The disclosure describes immunotoxin fusion proteins that address this unmet need.

### SUMMARY

It is against the above background that the present disclosure provides certain advantages over the prior art.

Although this disclosure is not limited to specific advantages or functionalities (such for example, the ability to make an immunotoxin fusion protein that can be used in methods of ablating hematopoietic stem cells in a patient in need thereof and methods of treatment of a patients with an acute myeloid leukemia (AML)), the disclosure provides an immunotoxin fusion protein, comprising:
(a) a bivalent single-chain fragment variable comprising two heavy chain variable domains and two light chain variable domains that specifically bind to the thrombopoietin receptor (cMPL); and
(b) a diphtheria toxin.

In one aspect of the immunotoxin fusion protein disclosed herein, the bivalent single-chain fragment variable has a structure represented by the formula:

V_{L1}-L₁-V_{H1}-L₂-V_{L2}-L₃-V_{H2}
wherein:
V_{L1} is a first light chain variable domain;
V_{H1} is a first a heavy chain variable domain;
V_{L2} is a second light chain variable domain;
V_{H2} is a second a heavy chain variable domain; and
L₁, L₂, are L₃ are each independently a linker domain or are absent.

In one aspect of the immunotoxin fusion protein disclosed herein, L₁, L₂, and L₃ comprise a peptide linker.

In one aspect of the immunotoxin fusion protein disclosed herein, the peptide linker is a 15-mer peptide linker comprising the amino acid sequence (G₄S)₃ (GGGGSGGGGSGGGGS; SEQ ID NO:14).

In one aspect of the immunotoxin fusion protein disclosed herein, the heavy chain variable domains each comprise an amino acid sequence having at least 90%, 91%, 92%, 93%, 94%, 95%, 96%, 97%, 98%, or 99% sequence identity to the amino acid sequence of SEQ ID NO: 5.

In one aspect of the immunotoxin fusion protein disclosed herein, the heavy chain variable domains each comprise the amino acid sequence of SEQ ID NO:5.

In one aspect of the immunotoxin fusion protein disclosed herein, the light chain variable domains each comprise an amino acid sequence having at least 90%, 91%, 92%, 93%, 94%, 95%, 96%, 97%, 98%, or 99% sequence identity to the amino acid sequence of SEQ ID NO: 4.

In one aspect of the immunotoxin fusion protein disclosed herein, the light chain variable domains each comprise the amino acid sequence of SEQ ID NO:4.

In one aspect of the immunotoxin fusion protein disclosed herein, the bivalent single-chain fragment variable comprises an amino acid sequence having at least 90%, 91%, 92%, 93%, 94%, 95%, 96%, 97%, 98%, or 99% sequence identity to the amino acid sequence of SEQ ID NO: 2.

In one aspect of the immunotoxin fusion protein disclosed herein, the bivalent single-chain fragment variable comprises the amino acid sequence of SEQ ID NO:2.

In one aspect of the immunotoxin fusion protein disclosed herein, the diphtheria toxin comprises an amino acid sequence having at least 90%, 91%, 92%, 93%, 94%, 95%, 96%, 97%, 98%, or 99% sequence identity to the amino acid sequence of SEQ ID NO: 17.

In one aspect of the immunotoxin fusion protein disclosed herein, the diphtheria toxin comprises an amino acid sequence having at least 90%, 91%, 92%, 93%, 94%, 95%, 96%, 97%, 98%, or 99% sequence identity to the amino acid sequence of SEQ ID NO:3.

In one aspect of the immunotoxin fusion protein disclosed herein, the diphtheria toxin comprises DT390.

In one aspect of the immunotoxin fusion protein disclosed herein, DT390 comprises the amino acid sequence of SEQ ID NO:3.

In one aspect of the immunotoxin fusion protein disclosed herein, the diphtheria toxin is operatively linked to the bivalent single-chain fragment variable at the N-terminus.

In one aspect of the immunotoxin fusion protein disclosed herein, the diphtheria toxin is operatively linked to the bivalent single-chain fragment variable at the C-terminus.

In one aspect of the immunotoxin fusion protein disclosed herein, the diphtheria toxin is operatively linked to the bivalent single-chain fragment variable by a linker (L₄).

In one aspect of the immunotoxin fusion protein disclosed herein, the linker (L₄) comprises a 5-mer peptide linker.

In one aspect of the immunotoxin fusion protein disclosed herein, the 5-mer peptide linker comprises the amino acid sequence G₄S (GGGGS; SEQ ID NO:15).

In one aspect of the immunotoxin fusion protein disclosed herein, the immunotoxin fusion protein comprises an amino acid sequence having at least 90%, 91%, 92%, 93%, 94%, 95%, 96%, 97%, 98%, or 99% sequence identity to the amino acid sequence of SEQ ID NO: 1.

In one aspect of the immunotoxin fusion protein disclosed herein, the immunotoxin fusion protein comprises the amino acid sequence of SEQ ID NO:1.

The invention also provides a polynucleotide encoding the immunotoxin fusion protein disclosed herein.

The invention also provides a vector, comprising the polynucleotide disclosed herein.

The invention also provides a host cell carrying the polynucleotide or the vector disclosed herein.

The invention also provides a pharmaceutical composition, comprising the immunotoxin fusion protein disclosed herein.

The invention also provides a method of ablating hematopoietic stem cells in a patient in need thereof, the method comprising administering to the patient an effective amount of the immunotoxin fusion protein or the pharmaceutical composition disclosed herein.

In one aspect of the methods disclosed herein, the patient is a hematopoietic stem cell transplantation recipient.

In one aspect of the methods disclosed herein, the method is performed before hematopoietic stem cell transplantation to the patient.

The invention also provides a method of treatment of a patients with an acute myeloid leukemia (AML), the method comprising administering to the patient an effective amount of the immunotoxin fusion protein or the pharmaceutical composition disclosed herein.

In one aspect of the methods disclosed herein, the AML is a type of a leukemia in which leukemic cells express the cMPL receptor.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows a schematic illustrating the structure of an immunotoxin fusion protein comprising DT390 and two scFV that specifically bind the thrombopoietin receptor (cMPL) (e.g., DT390-biscFV(cMPL)).

FIG. 2A-2F show the characterization of DT390-biscFV(cMPL) cytotoxic properties. FIG. 2A shows cMPL receptor-dependent cytotoxic effects of DT390-biscFV(cMPL) in a HEK293A cell line engineered to express the human cMPL receptor. FIG. 2B shows relative expression of cMPL in CD34+ cells, CD34+CD38− cells and phenotypically enriched CD34+CD38-CD90+CD45RA-CD49f+ hematopoietic stem cells (pHSCs). FIG. 2C shows human bulk CD34+ cells, and pHSCs cultured for 6 days in StemSpan medium supplemented with SCF, TPO, FLT3 and the indicated concentrations of DT390-biscFV(cMPL). FIG. 2D shows HSPC activity as determined by the frequency of human CD45+CD13+ myeloid cells in the peripheral blood of humanized immune-deficient mice monitored for 6 weeks. FIG. 2E shows that DT390-biscFV(cMPL) effectively depleted monkey HSCs in vitro. FIG. 2F shows that DT390-biscFV(cMPL) effectively depleted monkey HSCs in vivo.

FIG. 3 shows that DT390-biscFV(cMPL) has a favorable safety profile in vivo in monkeys.

FIG. 4 shows that the bivalent scFVs disclosed herein have a shorter half-life compared to related technologies (JSP-191 and MGTA-117 antibodies directed to CD117) allowing for faster cell infusion.

FIG. 5 shows a schematic illustrating the mechanism of action for DT390-biscFV(cMPL). Specifically, DT390-biscFV(cMPL) inhibits protein synthesis in HSCs causing cell death.

FIGS. 6A and 6B show the evaluation of the cytotoxic properties of DT390-biscFV(cMPL) in vivo. FIG. 6A shows representative flow cytometry plots showing cMPL⁺CD34⁺ cell populations in bone marrow aspirates of a rhesus macaque collected at baseline and 4 days after administration of 0.6 mg/kg DT390-biscFV(cMPL). FIG. 6B shows the frequency of cMPL-CD34⁺ long-term repopulating hematopoietic stem cells (LTR-HSCs) within bone marrow aspirates of rhesus macaques collected at baseline and on days 4, 18, and 56 days after administration of DT390-biscFV(cMPL) at various doses.

FIG. 7A-7D show the evaluation of the safety profile of DT390-biscFV(cMPL) in vivo. Peripheral blood counts and liver transaminase after treatment of rhesus macaques with DT390-biscFV(cMPL) at various doses. FIG. 7A is alanine transaminase. FIG. 7B is platelet counts. FIG. 7C is hemoglobin. FIG. 7D is neutrophil counts. Dotted horizontal line represent the normal range for each value measure.

FIG. 8 show the pharmacokinetic profile of DT3980-biscFV(cMPL) in vivo. Levels measured at 8 hours post treatment fell below the assay detection limit of 15 ng/ml (dotted line) at 0.2-0.6 mg/kg doses.

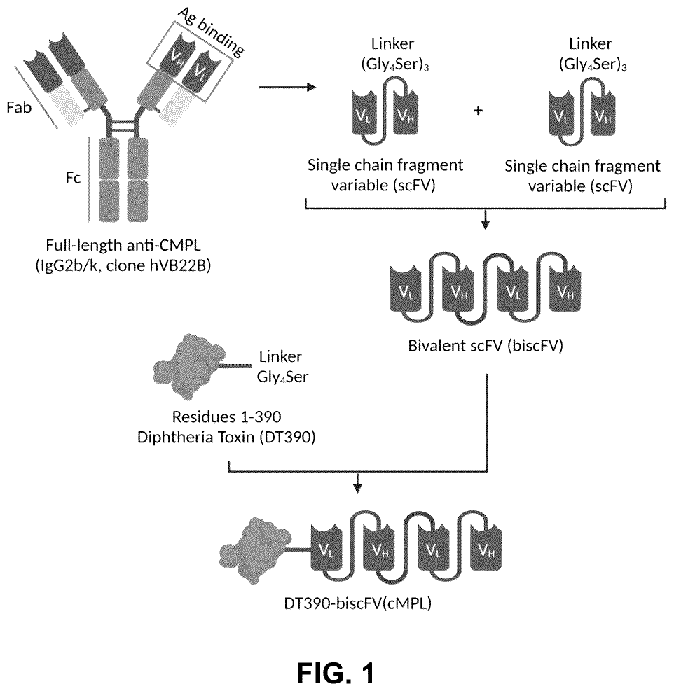

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiment(s) of the present invention.

### DETAILED DESCRIPTION

All publications, patents and patent applications cited herein are hereby expressly incorporated by reference for all purposes.

Before describing the present invention in detail, a number of terms will be defined. Unless otherwise required by context, singular terms shall include pluralities and plural terms shall include the singular. For example, the singular forms “a,” “an,” and “the” include plural referents unless the context clearly dictates otherwise.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

The terms “comprises” and variations thereof do not have a limiting meaning where these terms appear in the description and claims.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the term “about” is used to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. Ranges and amounts can be expressed as “about” a particular value or range. About can also include the exact amount. Typically, the term “about” includes an amount that would be expected to be within experimental error. The term “about” includes values that are within 10% less to 10% greater of the value provided.

As utilized in accordance with the present disclosure, unless otherwise indicated, all technical and scientific terms shall be understood to have the same meaning as commonly understood by one of ordinary skill in the art.

The disclosure relates to immunotoxin fusion proteins that comprise a bivalent single-chain fragment variable that specifically binds to the thrombopoietin receptor (cMPL) and a diphtheria toxin. The disclosure also provides compositions comprising such proteins and nucleic acid molecules encoding such proteins and uses thereof. The immunotoxin fusion proteins disclosed herein advantageously have enhanced affinity and specificity, and reduced off-target toxicity and half-life.

As used herein, and unless otherwise indicated, all technical and scientific terms shall be understood to have the same meaning as commonly understood by one of ordinary skill in the art. Unless otherwise required by context, singular terms shall include pluralities and plural terms shall include the singular.

Provided herein is an immunotoxin fusion protein comprising: (i) a bivalent single-chain fragment variable comprising two heavy chain variable domains and two light chain variable domains that specifically bind to the thrombopoietin receptor (cMPL); and (ii) a diphtheria toxin. As used herein, a “fusion protein” can comprise a first polypeptide (e.g., a biscFV-cMPL) operatively linked to a second polypeptide (e.g., a diphtheria toxin). Fusion proteins as disclosed herein may also optionally comprise a third, fourth or fifth or other polypeptide operatively linked to a first or second polypeptide. In certain embodiments, the fusion proteins as disclosed herein may also comprise one or more mutations in one or more of the polypeptides. Methods for making fusion proteins are well known in the art.

A “single-chain variable fragment” (sc (Fv)) includes a variable heavy chain domain (V_{H}) and a variable light chain domain (V_{L}) of an antibody, and these domains are present in a single polypeptide chain. The “Fv” fragment is the minimum antibody fragment and contains a complete antigen recognition site and a binding site. An “Fv” fragment is a dimer in which one V_{H} and V_{L} are tightly linked by non-covalent bonds (V_{H}-V_{L} dimer). The three complementarity-determining regions (CDRs) of each variable region interact to form an antigen-binding site on the surface of the V_{H}-V_{L} dimer. Six CDRs confer an antigen-binding site on the antibody.

Divalent (or bivalent) single-chain variable fragments (di-scFvs, bi-scFvs, sc(FV)₂) can be engineered by linking two scFvs. This can be done by producing a single peptide chain with two V_{H} and two V_{L} domains, yielding tandem scFvs. The order of the two VHS and the two V_{L}s may be arranged in any order.

In particular embodiments, disclosed herein is an immunotoxin fusion protein wherein the bivalent single-chain fragment variable has a structure represented by the formula:

V_{L1}-L₁-V_{H1}-L₂-V_{L2}-L₃-V_{H2}
wherein:
   V_{L1} is a first light chain variable domain;
   V_{H1} is a first a heavy chain variable domain;
   V_{L2} is a second light chain variable domain;
   V_{H2} is a second a heavy chain variable domain; and
   L₁, L₂, are L₃ are each independently a linker domain or are absent.

In particular embodiments, disclosed herein is an immunotoxin fusion protein wherein the bivalent single-chain fragment variable has a structure represented by the formula:

V_{L1}-L₁-V_{H1}-L₂-V_{H2}-L₃-V_{L2}
wherein:
   V_{L1} is a first light chain variable domain;
   V_{H1} is a first a heavy chain variable domain;
   V_{L2} is a second light chain variable domain;
   V_{H2} is a second a heavy chain variable domain; and
   L₁, L₂, are L₃ are each independently a linker domain or are absent.

In particular embodiments, disclosed herein is an immunotoxin fusion protein wherein the bivalent single-chain fragment variable has a structure represented by the formula:

V_{H1}-L₁-V_{L1}-L₂-V_{L2}-L₃-V_{H2}
wherein:
   V_{L1} is a first light chain variable domain;
   V_{H1} is a first a heavy chain variable domain;
   V_{L2} is a second light chain variable domain;
   V_{H2} is a second a heavy chain variable domain; and
   L₁, L₂, are L₃ are each independently a linker domain or are absent.

In particular embodiments, disclosed herein is an immunotoxin fusion protein wherein the bivalent single-chain fragment variable has a structure represented by the formula:

V_{H1}-L₁-V_{H2}-L₂-V_{L1}-L₃-V_{L2}
wherein:
   V_{L1} is a first light chain variable domain;
   V_{H1} is a first a heavy chain variable domain;
   V_{L2} is a second light chain variable domain;
   V_{H2} is a second a heavy chain variable domain; and
   L₁, L₂, are L₃ are each independently a linker domain or are absent.

In particular embodiments, disclosed herein is an immunotoxin fusion protein wherein the bivalent single-chain fragment variable has a structure represented by the formula:

V_{L1}-L₁-V_{L2}-L₂-V_{H1}-L₃-V_{H2}
wherein:
   V_{L1} is a first light chain variable domain;
   V_{H1} is a first a heavy chain variable domain;
   V_{L2} is a second light chain variable domain;
   V_{H2} is a second a heavy chain variable domain; and
   L₁, L₂, are L₃ are each independently a linker domain or are absent.

The term “linker” as used herein refers to one or more amino acid residues inserted between variable domains and/or between the bivalent single-chain fragment variable and the diphtheria toxin of the fusion proteins of the disclosure. For example, a linker may be inserted between two variable domains and/or between the bivalent single-chain fragment variable and the diphtheria toxin, at the sequence level. Linkers can comprise flexible amino acid residues (e.g., glycine or serine) to permit adjacent domains to move freely related to one another. The linkers L₁, L₂, L₃, and L₄, are independent, but in some embodiments of the fusion proteins of the disclosure may have the same sequence and/or length. In some embodiments, the amino acid composition of a linker can mimic the composition of linkers commonly found in recombinant proteins, which can generally by classified as flexible or rigid linkers. For example, flexible linkers found in recombinant proteins are generally composed of small, non-polar (e.g., Gly) or polar (e.g., Ser or Thr) amino acids whose small size provides flexibility and allows for mobility of the connecting functional domains. The incorporation of, e.g., Ser or Thr can maintain the stability of the linker in aqueous solutions by forming hydrogen bonds with the water molecules, and therefore can reduce interactions between the linker and the immunogens. In some embodiments, a linker comprises stretches of Gly and Ser residues (“GS” linker). Examples of widely used flexible linkers include, but are not limited to, (Gly-Gly-Ser)ₙ, (Gly-Gly-Gly-Ser), (SEQ ID NO: 16) or (Gly-Gly-Gly-Gly-Ser)ₙ (SEQ ID NO:15), where n=1-3. Adjusting the copy number “n” can optimize a linker to achieve sufficient separation of the functional domains. Many other flexible linkers have been designed for recombinant fusion proteins that can be used herein. In some embodiments, linkers can be rich in small or polar amino acids such as Gly and Ser, but also contain additional amino acids such as Thr and Ala to maintain flexibility, as well as polar amino acids such as Lys and Glu to improve solubility. In some embodiments, L₁, L₂ and L₃ comprise a 15-mer peptide linker. In some embodiments, the 15-mer peptide linker comprises the amino acid sequence (G₄S)₃ or (GGGGS)₃ (SEQ ID NO:14).

The terms “operably linked” or “operatively linked” refer to when a nucleotide sequence of interest is linked to one or more regulatory sequence(s) in a manner that allows for expression of the nucleotide sequence. In certain embodiments, operatively linked can refer to the one or more domains of a fusion protein being operatively linked in such a way that permits adjacent domains to move freely related to one another. The term “regulatory sequence” is intended to include, for example, promoters, enhancers and other expression control elements (e.g., polyadenylation signals). Such regulatory sequences are well known in the art and are described, for example, in Goeddel; Gene Expression Technology: Methods in Enzymology 185, Academic Press, San Diego, Calif. (1990). Regulatory sequences include those that direct constitutive expression of a nucleotide sequence in many types of host cells, and those that direct expression of the nucleotide sequence only in certain host cells (e.g., tissue-specific regulatory sequences). It will be appreciated by those skilled in the art that the design of the expression vector can depend on such factors as the choice of the target cell, the level of expression desired, and the like.

The term “antigen” or “target antigen” as used herein refers to a molecule or a portion of a molecule that is capable of being recognized by and bound by immunotoxin fusion proteins of the disclosure. A target antigen may have one or more epitopes.

A fusion protein is said to specifically bind an antigen when it preferentially recognizes its antigen target in a complex mixture of proteins and/or macromolecules. The term “specifically binds” refers to an immunotoxin fusion protein that specifically binds to a molecule or a fragment thereof (e.g., antigen). An immunotoxin fusion protein that specifically binds a molecule or a fragment thereof may bind to other molecules with lower affinity as determined by, for example, immunoassays, BIAcore, or other assays known in the art.

The term “K_{D},” as used herein, refers to the dissociation constant (K_{D}=[A]×[B]/[AB]) of the interaction between a fusion protein of the disclosure and an antigen target and has the units of moles/liter. An immunotoxin fusion protein of the disclosure typically has a dissociation constant (K_{D}) of 10⁻⁵ to 10⁻¹² moles/liter or less, or 10⁻⁷ to 10⁻¹² moles/liter or less, or 10⁻³ to 10⁻¹² moles/liter, and/or with a binding affinity of at least 10⁷ M⁻¹, or at least 10⁸ M⁻¹, or at least 10⁹ M⁻¹, or at least 10¹² M⁻¹. Any K_{D} value greater than 10⁻⁴ moles/liter is generally considered to indicate non-specific binding. Therefore, the lower the K_{D} value, the greater the affinity. In some embodiments, an immunotoxin fusion protein of the disclosure will bind to a desired antigen with an affinity less than 500 nM, or less than 200 nM, or less than 10 nM, or less than 500 pM. High affinity or very strong binding is often associated with greater efficacy, but it is not always the case that the greater the affinity the greater the efficacy.

The dissociation constant (K_{D}) can be determined, for example, by surface plasmon resonance (SPR). Generally, surface plasmon resonance analysis measures real-time binding interactions (both on rate and off rate) between a ligand (a target antigen on a biosensor matrix) and an analyte by surface plasmon resonance using, for example, the BIAcore system (Pharmacia Biosensor; Piscataway, NJ). Surface plasmon analysis can also be performed by immobilizing the analyte and presenting the ligand. Specific binding of immunotoxin fusion proteins of the disclosure to an antigen or antigenic determinant can also be determined in any suitable manner known in the art, including, for example, Scatchard analysis and/or competitive binding assays, such as radioimmunoassays (RIA), enzyme linked immunosorbent assays (ELISA), enzyme immunoassays (EIA), and sandwich competition assays.

In some embodiments, the bivalent single-chain fragment variable binds to the thrombopoietin receptor (cMPL). TPO receptor (cMPL) is a member of the hematopoietic receptor superfamily, which is a type I membrane protein having both conserved cysteine residues and a WSXWS box in the extracellular domain. Binding of TPO and cMPL triggers the homodimerization of receptors and transmits both proliferation and differentiation signals. In certain embodiments, the bivalent single-chain fragment variable comprises the sequences as disclosed in International Publication No. WO 2005056604, which is incorporated by reference herein in its entirety.

In some embodiments, the bivalent single-chain fragment variable comprises the amino acid sequence of SEQ ID NO:2. In some embodiments, the bivalent single-chain fragment variable comprises a heavy chain variable domain having an amino acid sequence that is at least 90, 91, 92, 93, 94, 95, 96, 97, 98, or 99% identical to the amino acid sequence of SEQ ID NO:5. In some embodiments, the bivalent single-chain fragment variable comprises a heavy chain variable domain having an amino acid sequence that is at least 90, 91, 92, 93, 94, 95, 96, 97, 98, or 99% identical to the amino acid sequence of any one of SEQ ID NO: 6-9. In some embodiments, the bivalent single-chain fragment variable comprises a light chain variable domain having an amino acid sequence that is at least 90, 91, 92, 93, 94, 95, 96, 97, 98, or 99% identical to the amino acid sequence of SEQ ID NO:4. In some embodiments, the bivalent single-chain fragment variable comprises a light chain variable domain having an amino acid sequence that is at least 90, 91, 92, 93, 94, 95, 96, 97, 98, or 99% identical to the amino acid sequence of any one of SEQ ID NO: 10-13.

As used herein, “percent (%) sequence identity” or “percent (%) identical” with respect to the polypeptide sequences identified herein is defined as the percentage of amino acid residues in a candidate sequence that are identical with the amino acid residues in the polypeptide being compared, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or Megalign (DNASTAR) software. Those skilled in the art can determine appropriate parameters for measuring alignment, including any algorithms needed to achieve maximal alignment over the full-length of the sequences being compared.

The immunotoxin fusion proteins disclosed herein further comprise a diphtheria toxin. In some embodiments, the diphtheria toxin comprises the amino acid sequence of SEQ ID NO: 17. In certain embodiments, the diphtheria toxin comprises an amino acid sequence that is at least 90, 91, 92, 93, 94, 95, 96, 97, 98, or 99% identical to the amino acid sequence of SEQ ID NO: 17. In some embodiments, the diphtheria toxin comprises a variant, mutant, and/or truncation of SEQ ID NO: 17. In some embodiments, a truncated diphtheria toxin can include DT386, DT387, DT388, DT389, DT390 or DT486. The diphtheria toxin can be conjugated to the bivalent single-chain fragment variable at the N-terminus or the C-terminus. In some embodiments, the diphtheria toxin is conjugated to the bivalent single-chain fragment variable by a linker (L₄). In some embodiments, the linker comprises a peptide linker. In some embodiments, the linker comprises a 5-mer peptide linker. In some embodiments, the 5-mer peptide linker comprises the amino acid sequence G₄S (GGGGS; SEQ ID NO:15).

In some embodiments, DT390 comprises an amino acid sequence that is at least 90, 91, 92, 93, 94, 95, 96, 97, 98, or 99% identical to the amino acid sequence of SEQ ID NO:3. In some embodiments, DT390 comprises the amino acid sequence of SEQ ID NO:3. In certain embodiments, the diphtheria toxin comprises DT390. DT390 is a truncated form of diphtheria toxin, which retains its enzyme activity and membrane translocation function, while deleting the binding domain to prevent its binding with normal cells, thereby diminishing its systemic toxicity. The diphtheria toxin can be conjugated to the bivalent single-chain fragment variable at the N-terminus or the C-terminus. In some embodiments, the diphtheria toxin is conjugated to the bivalent single-chain fragment variable by a linker (L₄). In some embodiments, the linker comprises a peptide linker. In some embodiments, the linker comprises a 5-mer peptide linker. In some embodiments, the 5-mer peptide linker comprises the amino acid sequence G₄S (GGGGS; SEQ ID NO:15).

In some embodiments, the immunotoxin fusion protein (DT390-biscFV(cMPL)) comprises an amino acid sequence that is at least 90, 91, 92, 93, 94, 95, 96, 97, 98, or 99% identical to the amino acid sequence of SEQ ID NO:1. In some embodiments, the immunotoxin fusion protein (DT390-biscFV(cMPL)) comprises the amino acid sequence of SEQ ID NO:1.

The term “vector,” refers to any molecule (e.g., nucleic acid, plasmid, or virus) that is used to transfer coding information to a host cell. One type of vector is a “plasmid,” which refers to a circular double-stranded DNA molecule into which additional DNA segments may be inserted. Another type of vector is a viral vector, wherein additional DNA segments may be inserted into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell and thereby are replicated along with the host genome. In addition, certain vectors are capable of directing the expression of genes to which they are operatively linked. Such vectors are referred to herein as “expression vectors.”

The term “host cell,” as used herein, refers to a cell into which an expression vector has been introduced. A host cell is intended to refer not only to the particular subject cell, but also to the progeny of such a cell. Because certain modifications may occur in succeeding generations due to either mutation or environmental influences, such progeny may not, in fact, be identical to the parent cell, but such cells are still included within the scope of the term “host cell” as used herein. A wide variety of host cell expression systems can be used to express the fusion proteins of the disclosure, including bacterial, yeast, baculoviral, and mammalian expression systems (as well as phage display expression systems).

One embodiment of the disclosure provides nucleic acid molecules comprising nucleotide sequences encoding the polypeptide chain that forms an immunotoxin fusion protein of the disclosure. Another embodiment of the disclosure provides expression vectors comprising nucleic acid molecules comprising nucleotide sequences encoding the polypeptide chain that forms the immunotoxin fusion proteins of the disclosure. Yet another embodiment of the disclosure provides host cells that express such immunotoxin fusion proteins (i.e., comprising nucleic acid molecules or vectors encoding the polypeptide chain that forms such immunotoxin fusion proteins).

The terms “pharmaceutical composition” or “therapeutic composition” as used herein refer to a compound or composition capable of inducing a desired therapeutic effect when properly administered to a patient. In some embodiments, the disclosure provides a pharmaceutical composition comprising a pharmaceutically acceptable carrier and a therapeutically effective amount of immunotoxin fusion proteins of the disclosure.

The terms “pharmaceutically acceptable carrier” or “physiologically acceptable carrier” as used herein refer to one or more formulation materials suitable for accomplishing or enhancing the delivery of one or more immunotoxin fusion proteins of the disclosure.

In some embodiments, the immunotoxin fusion proteins disclosed herein may be formulated with a pharmaceutically acceptable carrier, excipient, or stabilizer, as pharmaceutical compositions. In certain embodiments, such pharmaceutical compositions are suitable for administration to a human or non-human animal via any one or more routes of administration using methods known in the art. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also contain compatible solid or liquid fillers, diluents or encapsulating substances which are suitable for administration into a human. Other contemplated carriers, excipients, and/or additives, which may be utilized in the formulations described herein include, for example, flavoring agents, antimicrobial agents, sweeteners, antioxidants, antistatic agents, lipids, protein excipients such as serum albumin, gelatin, casein, salt-forming counterions such as sodium, and the like. These and additional known pharmaceutical carriers, excipients, and/or additives suitable for use in the formulations described herein are known in the art, for example, as listed in “Remington: The Science & Practice of Pharmacy,” 2Ist ed., Lippincott Williams & Wilkins, (2005), and in the “Physician's Desk Reference,” 60th ed., Medical Economics, Montvale, N.J. (2005). Pharmaceutically acceptable carriers can be selected that are suitable for the mode of administration, solubility, and/or stability desired or required.

In some embodiments, provided herein is a method of ablating hematopoietic stem cells in a patient in need thereof, the method comprising administering to the patient an effective amount of the immunotoxin fusion proteins disclosed herein. In some embodiments, the patient is a hematopoietic stem cell transplantation recipient. In some embodiments, the method, termed conditioning or preparative regimen, is performed before hematopoietic stem cell transplantation to the patient.

As used herein, the terms “treat,” “treatment,” or “treating” embrace at least an ablation and/or reduction of hematopoietic stem cells in a patient in need thereof, where ablation and/or reduction is used in a broad sense to refer to at least a reduction in the number of hematopoietic stem cells in the patient. As such, “treatment” also includes situations where the disease, disorder, or pathological condition, or at least symptoms associated therewith, are completely inhibited (e.g., prevented from happening) or stopped (e.g., terminated) such that the patient no longer suffers from the condition, or at least the symptoms that characterize the condition.

The term “patient” is intended to include human and non-human animals, particularly mammals. In some embodiments, mammals can include, but are not limited to, domesticated animals (e.g., cows, sheep, cats, dogs, and horses), primates (e.g., humans and non-human primates such as monkeys), rabbits, and rodents (e.g., mice and rats). In certain embodiments, the patient is a human.

The terms “administration” or “administering” as used herein refer to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

An “effective amount” of the fusion protein disclosed herein (or a pharmaceutical formulation), refers to an amount effective, at dosages and for periods of time necessary, to achieve the desired therapeutic or prophylactic result. In some embodiments, a therapeutically effective amount of the fusion protein administered to a patient will be in the range of about 0.01 to about 50 mg/kg of patient body weight, whether by one or more administrations. In some instances, the antibody used is about 0.01 mg/kg to about 45 mg/kg, about 0.01 mg/kg to about 40 mg/kg, about 0.01 mg/kg to about 35 mg/kg, about 0.01 mg/kg to about 30 mg/kg, about 0.01 mg/kg to about 25 mg/kg, about 0.01 mg/kg to about 20 mg/kg, about 0.01 mg/kg to about 15 mg/kg, about 0.01 mg/kg to about 10 mg/kg, about 0.01 mg/kg to about 5 mg/kg, about 0.2 mg/kg to about 0.8 mg/kg, or about 0.01 mg/kg to about 1 mg/kg administered daily, weekly, every two weeks, every three weeks, or monthly, for example. In some instances, the fusion protein is administered at about 0.2 mg/kg to about 0.8 mg/kg. However, other dosage regimens may be useful. In one instance, the fusion protein described herein is administered to a human at a dose of about 10 mg, about 20 mg, about 25 mg, about 30 mg, about 35 mg, about 40 mg, about 45 mg, about 50 mg, about 60 mg, about 70 mg, about 75 mg, about 80 mg, about 90 mg, about 100 mg, about 125 mg, about 150 mg, about 175 mg, about 200 mg, about 225 mg, about 250 mg, about 275 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg daily, weekly, every two weeks, every three weeks, or monthly. In some instances, the fusion protein is administered at about 45 mg intravenously daily, every two days, every three days, every four days, every five days, every six days, weekly, every two weeks, every three weeks, or monthly. The dose may be administered as a single dose or as multiple doses (e.g., 2 or 3 doses), such as infusions. In certain embodiments, the dose of the fusion protein administered in a combination treatment may be reduced as compared to a single treatment. The progress of this therapy is easily monitored by conventional techniques.

In some instances, the methods further involve administering to the patient an effective amount of an additional therapeutic agent. In some instances, the additional therapeutic agent is selected from the group consisting of an anti-neoplastic agent, a chemotherapeutic agent, a growth inhibitory agent, an anti-angiogenic agent, a radiation therapy, a cytotoxic agent, and combinations thereof.

Without limiting the disclosure, a number of embodiments of the disclosure are described herein for purpose of illustration.

### EXAMPLES

The Examples that follow are illustrative of specific embodiments of the disclosure, and various uses thereof. They are set forth for explanatory purposes only, and should not be construed as limiting the scope of the invention in any way.

### Example 1: Generation of Recombinant Bivalent Anti-cMPL Single-Chain Variable Fragment

Recombinant bivalent anti-cMPL single chain variable fragment contains two domains, diphtheria toxin truncated at residue 390 (DT390) to prevent toxin internalization in off-target cells (disclosed in Woo et al., (2002), Protein Expr. Purif. 25, 270-282) and two scFV (cMPL) fragments (disclosed in Orita et al., (2005), Blood 105; 562-566). A linker consisting of four glycines and a serine residue (G₄S) connected the DT390 domain to the two tandem scFV (cMPL) domains. The V_{L} and V_{H} domains of each scFV (cMPL) were linked by three tandem G₄S linkers [(G₄S)₃]. The two tandem scFV (cMPL) domains of DT390-biscFV(cMPL) were also joined by three tandem G₄S linkers [(G₄S)₃]. Six histidines (6×His tag) were added to the C-terminus to facilitate protein purification. The second scFV (cMPL) DNA (codon-optimized for yeast Pichia pastoris expression) was directly synthesized and cloned into pUC57 by GenScript. The first scFV (cMPL) DNA was amplified by PCR using the second scFV (cMPL) as PCR template. Both scFV (cMPL) domains were cloned into pwPICZalpha-DT390 vector (disclosed in Wang et al., (2011), Bioconjug Chem 22 (10), 2014-2020) yielding the final construct DT390-biscFV(cMPL) in pwPICZalpha. DT390-biscFV(cMPL) DNA construct was linearized and transformed into the diphtheria toxin-resistant yeast Pichia pastoris strain for protein expression (disclosed in Liu et al., (2003), Prot Expr Purif. 30(2), 262-274). Purification of DT390-biscFV(cMPL) was performed using an Ni-Sepharsoe™ 6 fast flow resin packed in an XK50 column (step 1), and a strong anion exchange resin Poros 50 HQ packed in an XK16/20 column (step 2). The resulting purified fusion protein is referred to as DT390-biscFV(cMPL).

### Example 2: Characterization of Recombinant Bivalent Anti-cMPL Single-Chain Variable Fragment (Sc(FV)₂)

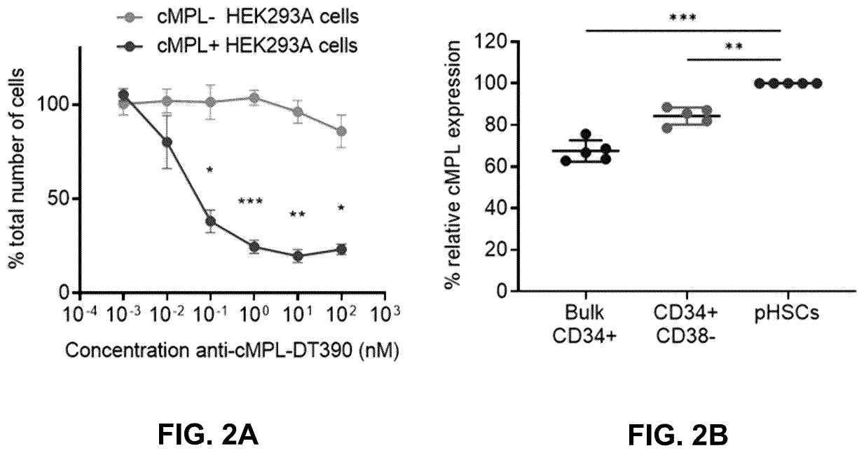

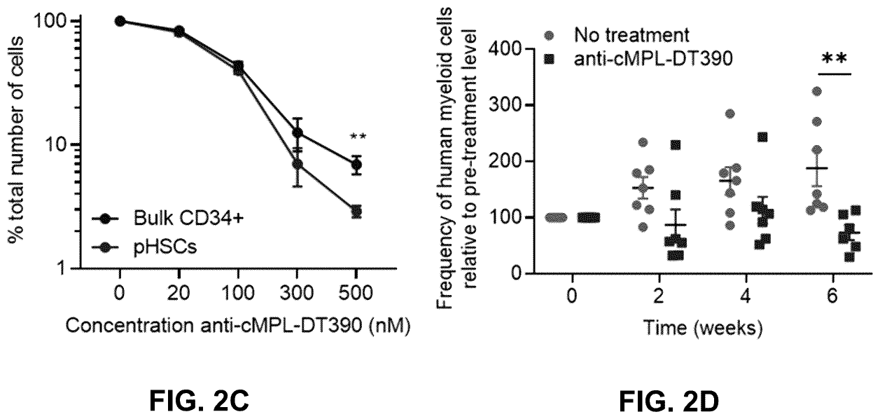

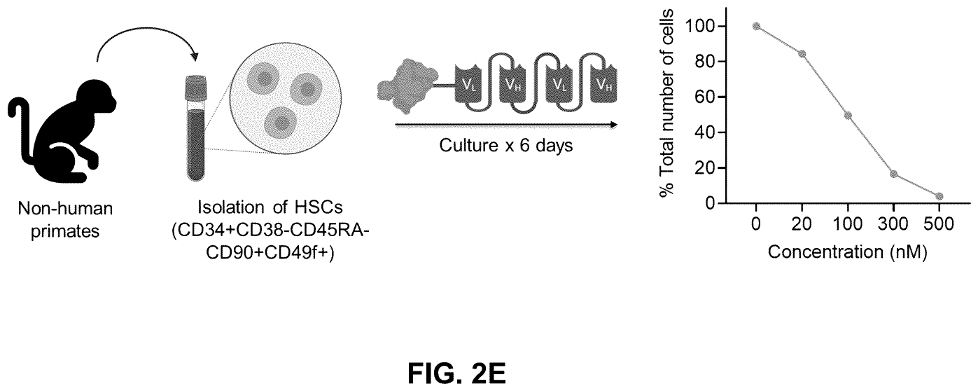

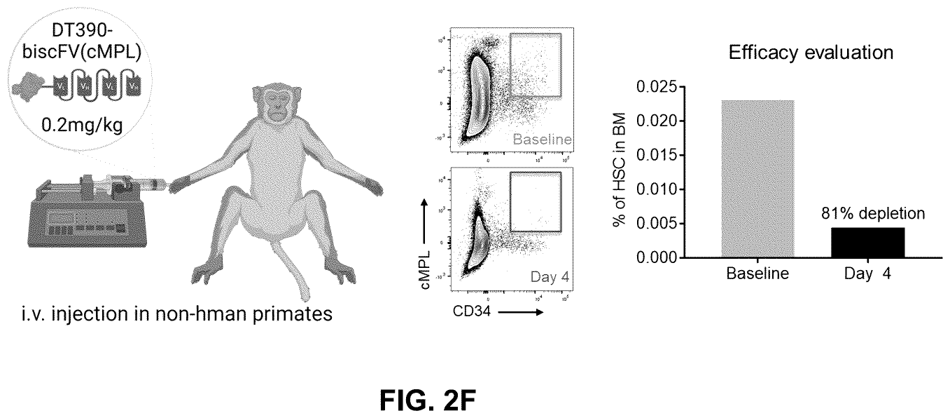

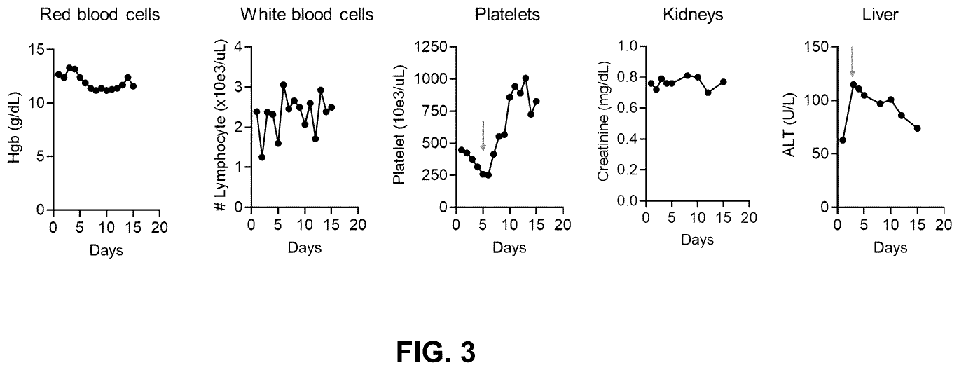

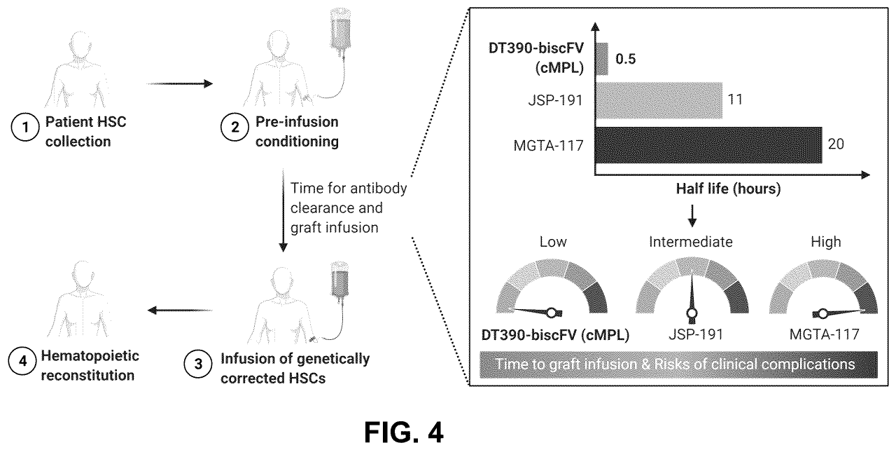

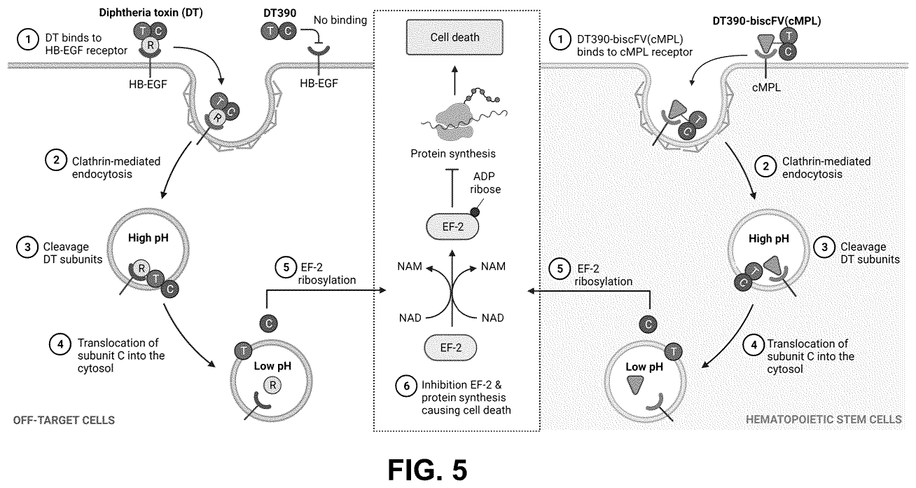

The cMPL receptor-dependent cytotoxic effects of DT390-biscFV(cMPL) fusion protein were investigated in a HEK293A cell line engineered to express the human cMPL receptor. Marked cellular killing in vitro (IC50=21 pM) was observed compared to the cMPL-negative control HEK293A cell line (FIG. 2A). Next, DT390-biscFV(cMPL) was assessed for its ability to inhibit growth of human CD34⁺ cells in vitro. G-CSF mobilized peripheral blood (PB) CD34⁺ cells were obtained from five healthy individuals. Surface expression of cMPL was compared by flow cytometry in subsets increasingly enriched in cells with long-term repopulating activity, including bulk CD34⁺, CD34⁺CD38⁻ and CD34⁺CD38⁻CD90⁺CD45RA⁻CD49f⁺ cells. Levels of cMPL expression increased congruently with levels of HSC purity (FIG. 2B). Consistent with a cMPL dependent cytotoxic effect, increased cellular death was measured in populations expressing higher densities of cMPL receptors (IC₅₀=104 nM), suggesting preferential targeting of the most primitive hematopoietic compartment (FIG. 2C). It was then assessed whether DT390-biscFV(cMPL) could safely target and deplete human HSPCs in vivo in humanized NBSGW immunodeficient mice. At 12 weeks post-transplantation, engrafted animals (mean 19.8% CD45+ cells in PB) received a single maximum tolerated dose of 1.2 mg/kg anti-CMPL-DT390 (n=7) or vehicle control solution (n=7) by tail vein injection. HSPC depletion was assayed by measuring human myeloid (CD45+CD13+) chimerism in the mouse PB after antibody administration. A gradual decline in HSPC activity was observed, as represented by the decreased production of human myeloid cells following administration of DT390-biscFV(cMPL), peaking at 6 weeks with a 2.6-fold reduction in frequency of human CD45+CD13+ cells compared to untreated animals (p=0.003) (FIG. 2D). Next, it was assessed whether DT390-biscFV(cMPL) could inhibit growth of highly purified non-human primate (rhesus macaques) HSPCs selected based on the cellular phenotype CD34⁺CD38⁻CD45RA⁻CD90⁺CD49f⁺. Similar to studies in human cells, a dose-dependent cytotoxic effect (IC50=145 nM) was observed in these cells after 6 days of culture (FIG. 2E). Finally, it was assessed whether DT390-biscFV(cMPL) could safely target and deplete rhesus macaque HSPCs in vivo. After a single intravenous injection of DT390-biscFV(cMPL) 0.2 mg/kg, a 10-fold selective reduction in marrow cMPL+CD34+ cells was observed (FIG. 2F). No significant systemic toxicity was observed at the dose tested (FIG. 3).

Overall, the example demonstrates that the bivalent anti-cMPL immunotoxins disclosed herein can effectively target and deplete human HSPCs.

### Example 3. Evaluation of the Efficacy of DT390-biscFV(cMPL) for HSPC Depletion In Vivo

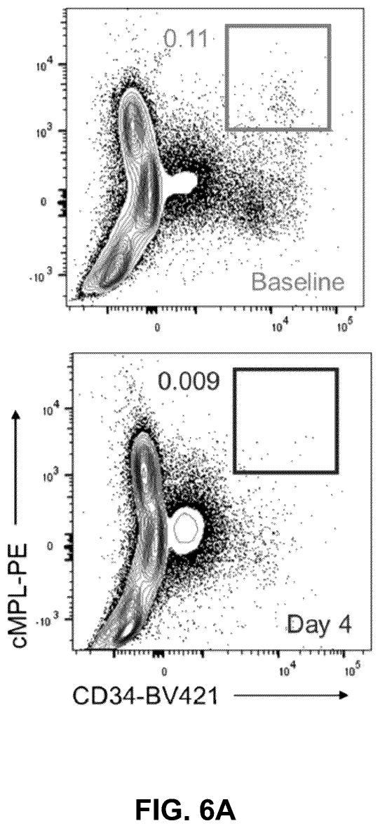

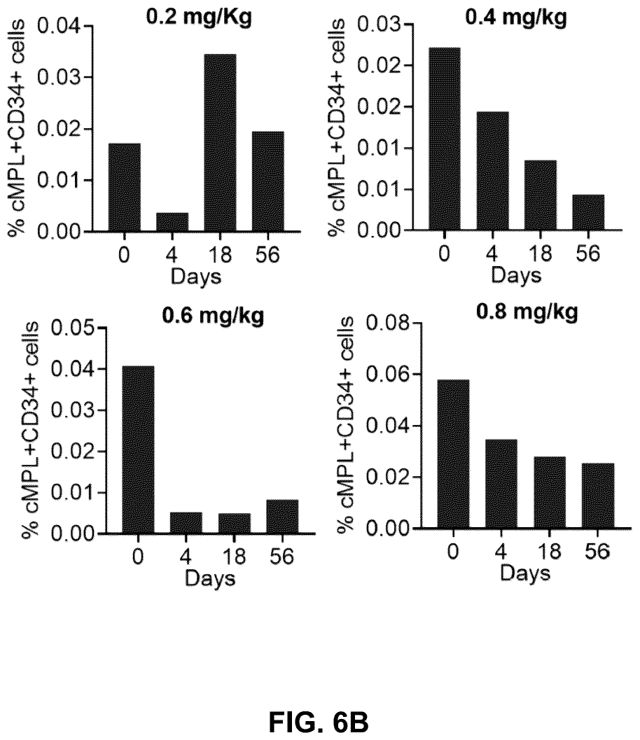

To assess whether DT390-biscFV(cMPL) can effectively deplete hematopoietic stem and progenitor cells (HSPCs) in vivo, single doses of 0.2, 0.4, 0.6 or 0.8 mg/kg of DT390-biscFV(cMPL) were administered into four independent rhesus macaques. HSPC depletion was measured by immunophenotyping of bone marrow aspirates collected pre- and post-treatment. Selective depletion of >90% was observed of the most primitive cMPL⁺CD34⁺ long-term repopulating hematopoietic stem cell (LTR-HSC) subset. The observed depletion persisted long-term at immunotoxin doses ≥0.4 mg/kg (FIGS. 6A and 6B). These data provide evidence that DT390-biscFV(cMPL) has potent cytotoxic activity against rhesus macaque LTR-HSCs in vivo.

### Example 4. Evaluation of the Safety of DT390-biscFV(cMPL) for HSPC Depletion In Vivo

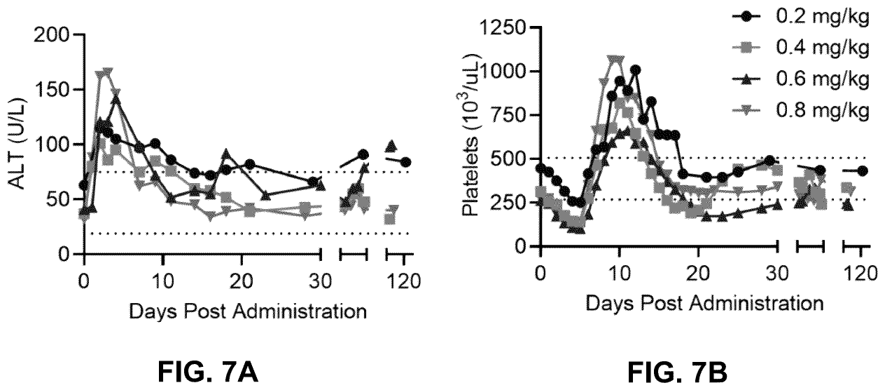

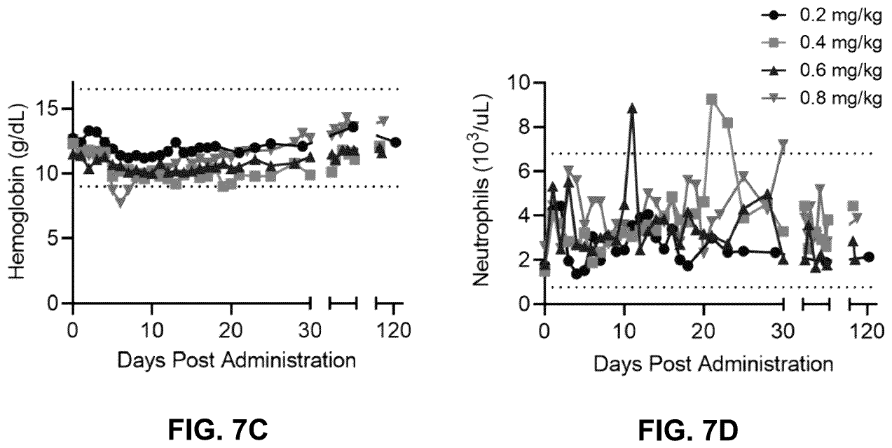

To evaluate the overall safety profile of DT390-biscFV(cMPL) in vivo, all animals were monitored closely via regular health checks (e.g., vital signs, physical examination) by the veterinary staff during and following immunotoxin administration. Complete blood counts (CBCs), chemistry and hepatic panels were obtained 3 times weekly for the first two weeks and once monthly thereafter. All animals remained well and active throughout the study. A transient (<5 days) 2- to 4-fold elevation in liver transaminases (FIG. 7A) was observed. A mild-to-moderate thrombocytopenia was also noted; no platelet transfusion was required and counts normalized within one week after treatment (FIG. 7B). Other hematopoietic lineages were largely unaffected (FIG. 7C, 7D). These data provide evidence that DT390-biscFV(cMPL) has a favorable safety profile in vivo.

### Example 5. Pharmacokinetics of DT390-biscFV(cMPL)

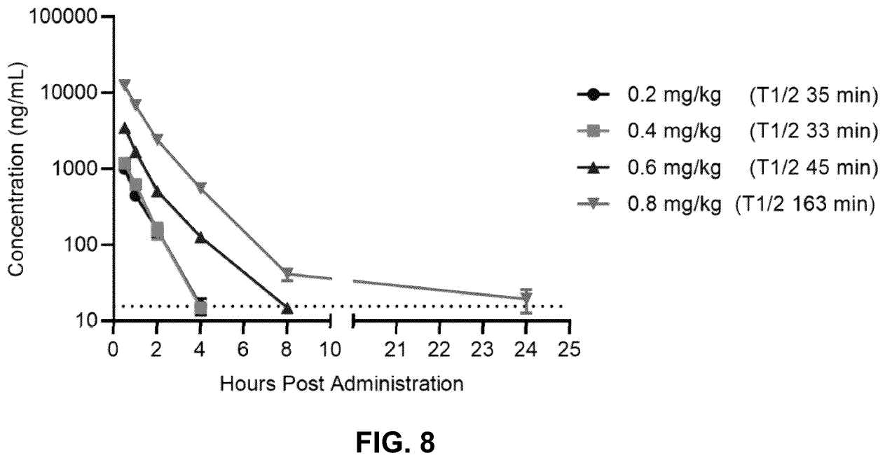

Because residual antibody in the serum of recipient animals would also deplete transplanted donor HSPCs, demonstration of a short serum half-life is critical for pre-transplant conditioning applications. The kinetics of antibody clearance for each dose tested (0.2 to 0.8 mg/kg) was determined by indirect ELISA assay. Notably, the drug displayed a short serum half-life (33-163 minutes) and was undetectable by 8-24 hours post-infusion, thus conferring a distinct advantage for pre-transplant conditioning applications (FIG. 8).

### Example 6. Evaluation of the Safety and Efficacy of DT390-biscFV(cMPL) for Pre-Transplant Conditioning

Experiments are done to evaluate DT390-biscFV(cMPL)-mediated elimination of endogenous HSPCs as a prospective conditioning regimen to enable safe engraftment of ex vivo gene-modified HSPCs to therapeutically meaningful levels. An autologous transplantation of genetically barcoded HSPCs was performed in rhesus macaques conditioned with a single intravenous infusion of 0.6 mg/kg DT390-biscFV(cMPL). Cells were re-infused 4 days after immunotoxin administration based on the peak efficacy of HSPC depletion post-treatment (FIG. 6 above) and the pharmacokinetics profile of DT390-biscFV(cMPL) (FIG. 8 above). To evaluate efficacy of this approach, the long-term stability of contributions from engrafted HSPC clones is examined by longitudinal follow-up of barcoded animals using previously described quantitative genetic barcoding methods. The clonal architecture is compared to historical data from rhesus macaques transplanted after conventional conditioning regimens (e.g., irradiation or busulfan). The safety profile is monitored long-term.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

TABLE 1
Sequences disclosed herein

SEQ ID NO: 1
DT390-biscFV(cMPL) (underlining identifies linkers)

| | |
|---|---|
| AGADDVVDSS KSFVMENFAS YHGTKPGYVD SIQKGIQKPK SGTOGNYDDD WKGFYSTDNK | 60 |
| YDAAGYSVDN ENPLSGKAGG VVKVTYPGLT KVLALKVDNA ETIKKELGLS LTEPLMEQVG | 120 |
| TEEFIKRFGD GASRVVLSLP FAEGSSSVEY INNWEQAKAL SVELEINFET RGKRGQDAMY | 180 |
| EYMAQACAGN RVRRSVGSSL SCINLDWDVI RDKTKTKIES LKEHGPIKNK MSESPAKTVS | 240 |
| EEKAKQYLEE FHQTALEHPE LSELKTVTGT NPVFAGANYA AWAVNVAQVI DSETADNLEK | 300 |
| TTAALSILPG IGSVMGIADG AVHHNTEEIV AQSIALSSLM VAQAIPLVGE LVDIGFAAYN | 360 |
| FVESIINLFQ VVHNSYNRPA YSPGHKTQPF LPWGGGGSDI VMTQSALSLP VTPGEPASIS | 420 |
| CRSSKSLLHS NGNTYLYWYL QKPGOSPOLL IYRMSNLASG VPDRFSGSGS GTAFTLKISR | 480 |
| VEAEDVGVYY CMQHIEYPFT FGQGTKLEIK GGGGSGGGGS GGGGSQVOLV QSGPEVKKPG | 540 |
| ASVKVSCKAS GYTFTNSWMN WVRQRPGKGL EWVGRIYPGD GETIYNGKFR VRVTITADES | 600 |
| TSTAYMELSS LRSEDTAVYY CARGYDDYSF AYWGQGTTVT VSSGGGGSGG GGSGGGGSDI | 660 |
| VMTQSALSLP VTPGEPASIS CRSSKSLLHS NGNTYLYWYL QKPGOSPQLL IYRMSNLASG | 720 |
| VPDRFSGSGS GTAFTLKISR VEAEDVGVYY CMQHIEYPFT FGQGTKLEIK GGGGSGGGGS | 780 |
| GGGGSQVQLV QSGPEVKKPG ASVKVSCKAS GYTFTNSWMN WVRQRPGKGL EWVGRIYPGD | 840 |
| GETIYNGKFR VRVTITADES TSTAYMELSS LRSEDTAVYY CARGYDDYSF AYWGQGTTVT | 900 |
| VSSHHHHHH | 909 |

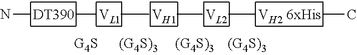

SEQ ID NO: 2
biscFV(cMPL) in DT390-biscFV(cMPL)

| | |
|---|---|
| DIVMTQSALS LPVTPGEPAS ISCRSSKSLL HSNGNTYLYW YLQKPGQSPQ LLIYRMSNLA | 60 |
| SGVPDRFSGS GSGTAFTLKI SRVEAEDVGV YYCMQHIEYP FTFGQGTKLE IKGGGGSGGG | 120 |
| GSGGGGSQVQ LVQSGPEVKK PGASVKVSCK ASGYTFTNSW MNWVRQRPGK GLEWVGRIYP | 180 |
| GDGETIYNGK FRVRVTITAD ESTSTAYMEL SSLRSEDTAV YYCARGYDDY SFAYWGOGTT | 240 |
| VTVSSGGGGS GGGGSGGGGS DIVMTOSALS LPVTPGEPAS ISCRSSKSLL HSNGNTYLYW | 300 |
| YLQKPGQSPQ LLIYRMSNLA SGVPDRFSGS GSGTAFTLKI SRVEAEDVGV YYCMQHIEYP | 360 |
| FTFGQGTKLE IKGGGGSGGG GSGGGGSQVQ LVQSGPEVKK PGASVKVSCK ASGYTFTNSW | 420 |
| MNWVRQRPGK GLEWVGRIYP GDGETIYNGK FRVRVTITAD ESTSTAYMEL SSLRSEDTAV | 480 |
| YYCARGYDDY SFAYWGOGTT VTVSS | 505 |

SEQ ID NO: 3
DT390 in DT390-biscFV(cMPL)

| | |
|---|---|
| AGADDVVDSS KSFVMENFAS YHGTKPGYVD SIQKGIQKPK SGTQGNYDDD WKGFYSTDNK | 60 |
| YDAAGYSVDN ENPLSGKAGG VVKVTYPGLT KVLALKVDNA ETIKKELGLS LTEPLMEQVG | 120 |
| TEEFIKRFGD GASRVVLSLP FAEGSSSVEY INNWEQAKAL SVELEINFET RGKRGQDAMY | 180 |
| EYMAQACAGN RVRRSVGSSL SCINLDWDVI RDKTKTKIES LKEHGPIKNK MSESPAKTVS | 240 |
| EEKAKQYLEE FHQTALEHPE LSELKTVTGT NPVFAGANYA AWAVNVAQVI DSETADNLEK | 300 |
| TTAALSILPG IGSVMGIADG AVHHNTEEIV AQSIALSSLM VAQAIPLVGE LVDIGFAAYN | 360 |
| FVESIINLFQ VVHNSYNRPA YSPGHKTQPF LPW | 393 |

SEQ ID NO: 4
Light chain variable domains (V_{L1} and V_{L2}) in DT390-biscFV(cMPL)

| | |
|---|---|
| DIVMTQSALS LPVTPGEPAS ISCRSSKSLL HSNGNTYLYW YLQKPGQSPQ LLIYRMSNLA | 60 |
| SGVPDRFSGS GSGTAFTLKI SRVEAEDVGV YYCMQHIEYP FTFGOGTKLE IK | 112 |

SEQ ID NO: 5
Heavy chain variable domains (VH1 and VH2) in DT390-biscFV(cMPL)

| | |
|---|---|
| QVQLVQSGPE VKKPGASVKV SCKASGYTFT NSWMNWVRQR PGKGLEWVGR IYPGDGETIY | 60 |
| NGKFRVRVTI TADESTSTAY MELSSLRSED TAVYYCARGY DDYSFAYWGQ GTTVTVSS | 118 |

SEQ ID NO: 6
Heavy chain variable domain in hVB22B u2-wz4

| | |
|---|---|
| QVQLVQSGPE VKKPGASVKV SCKASGYTFT NSWMNWVRQR PGKGLEWIGR IYPGDGETIY | 60 |
| NGKFRVRVTI TADESTSTAY MQLSSLRSED TAVYYCARGY DDYSFAYWGQ GTTVTVSS | 118 |

SEQ ID NO: 7
Heavy chain variable domain in hVB22B q-wz5

| | |
|---|---|
| QVQLVQSGPE VKKPGASVKV SCKASGYTFT NSWMNWVRQR PGKGLEWIGR IYPGDGETIY | 60 |
| NGKFRVRVTI TADESTSTAY MELSSLRSED TAVYYCARGY DDYSFAYWGQ GTTVTVSS | 118 |

SEQ ID NO: 8
Heavy chain variable domain) in hVB22B p-z

| | |
|---|---|
| QVQLVQSGPE VKKPGASVKV SCKASGYTFT NSWMNWVRQR PGKGLEWMGR IYPGDGETIY | 60 |
| NGKFRVRVTI TADESTSTAY MELSSLRSED TAVYYCARGY DDYSFAYWGQ GTTVTVSS | 118 |

SEQ ID NO: 9
Heavy chain variable domain in hVB22B e

| | |
|---|---|
| QVQLVQSGPE VKKPGASVKV SCKASGYTFT NSWMNWIRQR PGKGLEWIGR IYPGDGETIY | 60 |
| NGKFRVRVTI TADESTSTAY MELSSLRSED TAVYYCARGY DDYSFAYWGQ GTLVTVSS | 118 |

SEQ ID NO: 10
Light chain variable domain in hVB22B u2-wz4

| | |
|---|---|
| DIVMTQSPLS LPVTPGEPAS ISCRSSKSLL HSNGNTYLYW FLOKPGQSPQ LLIYRMSNLA | 60 |
| SGVPDRFSGS GSGTDFTLKI SRVEAEDVGV YYCMQHIEYP FTFGQGTKLE IK | 112 |

SEQ ID NO:11
Light chain variable domain in hVB22B q-wz5

| | |
|---|---|
| DIVMTQSPLS LPVTPGEPAS ISCRSSKSLL HSNGNTYLYW FQQKPGQAPR LLIYRMSNLA | 60 |
| SGVPDRFSGS GSGTAFTLKI SRVEAEDVGV YYCMQHIEYP FTFGQGTKLE IK | 112 |

SEQ ID NO: 12
Light chain variable domain in hVB22B p-z

| | |
|---|---|
| DIVMTQSALS LPVTPGEPAS ISCRSSKSLL HSNGNTYLYW FQQKPGOSPQ LLIYRMSNLA | 60 |
| SGVPDRFSGS GSGTAFTLKI SRVEAEDVGV YYCMQHIEYP FTFGQGTKLE IK | 112 |

SEQ ID NO: 13
Light chain variable domain in hVB22B e

| | |
|---|---|
| DIVMTQSALS LPVTPGEPAS ISCRSSKSLL HSNGNTYLYW YLQKPGOSPQ LLIYRMSNLA | 60 |
| SGVPDRFSGS GSGTAFTLKI SRVEAEDVGV YYCMQHIEYP FTFGOGTKLE IK | 112 |

SEQ ID NO: 14
(G4S)3 linker
GGGGSGGGGSGGGGS
SEQ ID NO: 15
(G4S) linker
GGGGS
SEQ ID NO: 16
(G3S) linker
GGGS
SEQ ID NO: 17
Diphtheria toxin, Corynebacterium diphtheriae (Q6NK15)
MSRKLFASILIGALLGIGAPPSAHAGADDVVDSSKSFVMENFSSYHGTKPGYVDSIQKGI
QKPKSGTQGNYDDDWKGFYSTDNKYDAAGYSVDNENPLSGKAGGVVKVTYPGLTKVLALK
VDNAETIKKELGLSLTEPLMEQVGTEEFIKRFGDGASRVVLSLPFAEGSSSVEYINNWEQ
AKALSVELEINFETRGKRGQDAMYEYMAQACAGNRVRRSVGSSLSCINLDWDVIRDKTKT
KIESLKEHGPIKNKMSESPNKTVSEEKAKQYLEEFHQTALEHPELSELKTVTGTNPVFAG
ANYAAWAVNVAQVIDSETADNLEKTTAALSILPGIGSVMGIADGAVHHNTEEIVAQSIAL
SSLMVAQAIPLVGELVDIGFAAYNFVESIINLFQVVHNSYNRPAYSPGHKTQPFLHDGYA
VSWNTVEDSIIRTGFQGESGHDIKITAENTPLPIAGVLLPTIPGKLDVNKSKTHISVNGR
KIRMRCRAIDGDVTFCRPKSPVYVGNGVHANLHVAFHRSSSEKIHSNEISSDSIGVLGYQ
KTVDHTKVNSKLSLFFEIKS

What is claimed is:

1. An immunotoxin fusion protein, comprising:
(a) a bivalent single-chain fragment variable comprising two heavy chain variable domains and two light chain variable domains that specifically bind to the thrombopoietin receptor (cMPL); and
(b) a diphtheria toxin.

2. The immunotoxin fusion protein of claim 1, wherein the bivalent single-chain fragment variable has a structure represented by the formula:
V_{L1}-L₁-V_{H1}-L₂-V_{L2}-L₃-V_{H2}
wherein:
V_{L1} is a first light chain variable domain;
V_{H1} is a first a heavy chain variable domain;
V_{L2} is a second light chain variable domain;
V_{H2} is a second a heavy chain variable domain; and
L₁, L₂, are L₃ are each independently a linker domain or are absent.

3. The immunotoxin fusion protein of claim 2, wherein L₁, L₂, and L₃ comprise a peptide linker.

4. The immunotoxin fusion protein of claim 3, wherein the peptide linker is a 15-mer peptide linker comprising the amino acid sequence (GGGGS)₃ (SEQ ID NO:14).

5. The immunotoxin fusion protein of claim 1, wherein the heavy chain variable domains each comprise an amino acid sequence having at least 90%, 91%, 92%, 93%, 94%, 95%, 96%, 97%, 98%, or 99% sequence identity to the amino acid sequence of SEQ ID NO:5.

6. The immunotoxin fusion protein of claim 1, wherein the light chain variable domains each comprise an amino acid sequence having at least 90%, 91%, 92%, 93%, 94%, 95%, 96%, 97%, 98%, or 99% sequence identity to the amino acid sequence of SEQ ID NO:4.

7. The immunotoxin fusion protein of claim 1, wherein the bivalent single-chain fragment variable comprises an amino acid sequence having at least 90%, 91%, 92%, 93%, 94%, 95%, 96%, 97%, 98%, or 99% sequence identity to the amino acid sequence of SEQ ID NO: 2.

8. The immunotoxin fusion protein of any one of claims 1-7, wherein the diphtheria toxin comprises an amino acid sequence having at least 90%, 91%, 92%, 93%, 94%, 95%, 96%, 97%, 98%, or 99% sequence identity to the amino acid sequence of SEQ ID NO: 17.

9. The immunotoxin fusion protein of claim 8, wherein the diphtheria toxin comprises an amino acid sequence having at least 90%, 91%, 92%, 93%, 94%, 95%, 96%, 97%, 98%, or 99% sequence identity to the amino acid sequence of SEQ ID NO:3.

10. The immunotoxin fusion protein of any one of claims 1-9, wherein the diphtheria toxin is operatively linked to the bivalent single-chain fragment variable at the N-terminus.

11. The immunotoxin fusion protein of any one of claims 1-9, wherein the diphtheria toxin is operatively linked to the bivalent single-chain fragment variable at the C-terminus.

12. The immunotoxin fusion protein of any one of claims 1-11, wherein the diphtheria toxin is operatively linked to the bivalent single-chain fragment variable by a linker (L₄).

13. The immunotoxin fusion protein of claim 12, wherein the linker (L₄) comprises a peptide linker.

14. The immunotoxin fusion protein of claim 13, wherein the peptide linker is a 5-mer peptide linker comprising the amino acid sequence GGGGS (SEQ ID NO: 15).

15. The immunotoxin fusion protein of any one of claims 1-14, wherein the immunotoxin fusion protein comprises an amino acid sequence having at least 90%, 91%, 92%, 93%, 94%, 95%, 96%, 97%, 98%, or 99% sequence identity to the amino acid sequence of SEQ ID NO: 1.

16. A polynucleotide encoding the immunotoxin fusion protein of any one of claims 1-15.

17. A vector, comprising the polynucleotide of claim 16.

18. A host cell carrying the polynucleotide of claim 16 or the vector of claim 17.

19. A pharmaceutical composition, comprising the immunotoxin fusion protein of any one of claims 1-15.

20. A method of ablating hematopoietic stem cells in a patient in need thereof, the method comprising administering to the patient an effective amount of the immunotoxin fusion protein of any one of claims 1-15, or the pharmaceutical composition of claim 19.

21. The method of claim 20, wherein the patient is a hematopoietic stem cell transplantation recipient.

22. The method of claim 21, wherein the method is performed before hematopoietic stem cell transplantation to the patient.

23. A method of treatment of a patient with an acute myeloid leukemia (AML), the method comprising administering to the patient an effective amount of the immunotoxin fusion protein of any one of claims 1-15, or the pharmaceutical composition of claim 19.

24. The method of claim 23, wherein the AML is a type of a leukemia in which leukemic cells express the cMPL receptor.


SEQUENCE LISTING (17 sequences)

SEQ ID NO: 1 - protein, 909 aa, synthetic construct
  REGION 1..909: note = Synthetic peptide
AGADDVVDSSKSFVMENFASYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPAKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLPWGGGGSDIVMTQSALSLPVTPGEPASISCRSSKSLLHSNGNTYLYWYLQKPGQSPQLLIYRMSNLASGVPDRFSGSGSGTAFTLKISRVEAEDVGVYYCMQHIEYPFTFGQGTKLEIKGGGGSGGGGSGGGGSQVQLVQSGPEVKKPGASVKVSCKASGYTFTNSWMNWVRQRPGKGLEWVGRIYPGDGETIYNGKFRVRVTITADESTSTAYMELSSLRSEDTAVYYCARGYDDYSFAYWGQGTTVTVSSGGGGSGGGGSGGGGSDIVMTQSALSLPVTPGEPASISCRSSKSLLHSNGNTYLYWYLQKPGQSPQLLIYRMSNLASGVPDRFSGSGSGTAFTLKISRVEAEDVGVYYCMQHIEYPFTFGQGTKLEIKGGGGSGGGGSGGGGSQVQLVQSGPEVKKPGASVKVSCKASGYTFTNSWMNWVRQRPGKGLEWVGRIYPGDGETIYNGKFRVRVTITADESTSTAYMELSSLRSEDTAVYYCARGYDDYSFAYWGQGTTVTVSSHHHHHH

SEQ ID NO: 2 - protein, 505 aa, synthetic construct
  REGION 1..505: note = Synthetic peptide
DIVMTQSALSLPVTPGEPASISCRSSKSLLHSNGNTYLYWYLQKPGQSPQLLIYRMSNLASGVPDRFSGSGSGTAFTLKISRVEAEDVGVYYCMQHIEYPFTFGQGTKLEIKGGGGSGGGGSGGGGSQVQLVQSGPEVKKPGASVKVSCKASGYTFTNSWMNWVRQRPGKGLEWVGRIYPGDGETIYNGKFRVRVTITADESTSTAYMELSSLRSEDTAVYYCARGYDDYSFAYWGQGTTVTVSSGGGGSGGGGSGGGGSDIVMTQSALSLPVTPGEPASISCRSSKSLLHSNGNTYLYWYLQKPGQSPQLLIYRMSNLASGVPDRFSGSGSGTAFTLKISRVEAEDVGVYYCMQHIEYPFTFGQGTKLEIKGGGGSGGGGSGGGGSQVQLVQSGPEVKKPGASVKVSCKASGYTFTNSWMNWVRQRPGKGLEWVGRIYPGDGETIYNGKFRVRVTITADESTSTAYMELSSLRSEDTAVYYCARGYDDYSFAYWGQGTTVTVSS

SEQ ID NO: 3 - protein, 393 aa, synthetic construct
  REGION 1..393: note = Synthetic peptide
AGADDVVDSSKSFVMENFASYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPAKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLPW

SEQ ID NO: 4 - protein, 112 aa, synthetic construct
  REGION 1..112: note = Synthetic peptide
DIVMTQSALSLPVTPGEPASISCRSSKSLLHSNGNTYLYWYLQKPGQSPQLLIYRMSNLASGVPDRFSGSGSGTAFTLKISRVEAEDVGVYYCMQHIEYPFTFGQGTKLEIK

SEQ ID NO: 5 - protein, 118 aa, synthetic construct
  REGION 1..118: note = Synthetic peptide
QVQLVQSGPEVKKPGASVKVSCKASGYTFTNSWMNWVRQRPGKGLEWVGRIYPGDGETIYNGKFRVRVTITADESTSTAYMELSSLRSEDTAVYYCARGYDDYSFAYWGQGTTVTVSS

SEQ ID NO: 6 - protein, 118 aa, synthetic construct
  REGION 1..118: note = Synthetic peptide
QVQLVQSGPEVKKPGASVKVSCKASGYTFTNSWMNWVRQRPGKGLEWIGRIYPGDGETIYNGKFRVRVTITADESTSTAYMQLSSLRSEDTAVYYCARGYDDYSFAYWGQGTTVTVSS

SEQ ID NO: 7 - protein, 118 aa, synthetic construct
  REGION 1..118: note = Synthetic peptide
QVQLVQSGPEVKKPGASVKVSCKASGYTFTNSWMNWVRQRPGKGLEWIGRIYPGDGETIYNGKFRVRVTITADESTSTAYMELSSLRSEDTAVYYCARGYDDYSFAYWGQGTTVTVSS

SEQ ID NO: 8 - protein, 118 aa, synthetic construct
  REGION 1..118: note = Synthetic peptide
QVQLVQSGPEVKKPGASVKVSCKASGYTFTNSWMNWVRQRPGKGLEWMGRIYPGDGETIYNGKFRVRVTITADESTSTAYMELSSLRSEDTAVYYCARGYDDYSFAYWGQGTTVTVSS

SEQ ID NO: 9 - protein, 118 aa, synthetic construct
  REGION 1..118: note = Synthetic peptide
QVQLVQSGPEVKKPGASVKVSCKASGYTFTNSWMNWIRQRPGKGLEWIGRIYPGDGETIYNGKFRVRVTITADESTSTAYMELSSLRSEDTAVYYCARGYDDYSFAYWGQGTLVTVSS

SEQ ID NO: 10 - protein, 112 aa, synthetic construct
  REGION 1..112: note = Synthetic peptide
DIVMTQSPLSLPVTPGEPASISCRSSKSLLHSNGNTYLYWFLQKPGQSPQLLIYRMSNLASGVPDRFSGSGSGTDFTLKISRVEAEDVGVYYCMQHIEYPFTFGQGTKLEIK

SEQ ID NO: 11 - protein, 112 aa, synthetic construct
  REGION 1..112: note = Synthetic peptide
DIVMTQSPLSLPVTPGEPASISCRSSKSLLHSNGNTYLYWFQQKPGQAPRLLIYRMSNLASGVPDRFSGSGSGTAFTLKISRVEAEDVGVYYCMQHIEYPFTFGQGTKLEIK

SEQ ID NO: 12 - protein, 112 aa, synthetic construct
  REGION 1..112: note = Synthetic peptide
DIVMTQSALSLPVTPGEPASISCRSSKSLLHSNGNTYLYWFQQKPGQSPQLLIYRMSNLASGVPDRFSGSGSGTAFTLKISRVEAEDVGVYYCMQHIEYPFTFGQGTKLEIK

SEQ ID NO: 13 - protein, 112 aa, synthetic construct; identical to SEQ ID NO: 4
  REGION 1..112: note = Synthetic peptide

SEQ ID NO: 14 - protein, 15 aa, synthetic construct
  REGION 1..15: note = Synthetic peptide
GGGGSGGGGSGGGGS

SEQ ID NO: 15 - protein, 5 aa, synthetic construct
  REGION 1..5: note = Synthetic peptide
GGGGS

SEQ ID NO: 16 - protein, 4 aa, synthetic construct
  REGION 1..4: note = Synthetic peptide
GGGS

SEQ ID NO: 17 - protein, 560 aa, Corynebacterium diphtheriae
MSRKLFASILIGALLGIGAPPSAHAGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS